(19) United States
(12) Patent Application Publication
(10) Pub. No.: US 2023/0079301 A1
(43) Pub. Date: Mar. 16, 2023

(54) DEVICE FOR DIAGNOSING MALE INFERTILITY

(71) Applicant: PERJEY LLC, Chicago, IL (US)

(72) Inventors: Rajasingam S. JEYENDRAN, Lisle, IL (US); Eugene PERGAMENT, Chicago, IL (US)

(21) Appl. No.: 17/889,439

(22) Filed: Aug. 17, 2022

Related U.S. Application Data
(60) Provisional application No. 63/239,483, filed on Sep. 1, 2021.

(51) Int. Cl. G01N 27/406; G01N 33/50

(52) U.S. Cl. CPC G01N 27/4067; G01N 33/5044

(57) ABSTRACT

Diagnostic devices for diagnosing male infertility and associated methods are described herein. In one aspect, a diagnostic device can include a thermally conductive probe configured to contact a Scrotal-Testes (ST) Complex of a patient; a thermally insulative housing detachably coupled to the thermally conductive probe; and a base defining a cavity, the base including: a thermal sensor positioned within the cavity and detachably coupled to a bottom surface of the thermally conductive probe; at least one processor in electronic communication with the thermal sensor and positioned within the cavity; and a receptacle formed by an exterior surface of the base and detachably coupled to the thermally insulative housing.

### CROSS-REFERENCE TO RELATED APPLICATION

This application claims priority under Title 35, United States Code, Section 119(e), to U.S. Provisional Application Ser. No. 63/239,483 titled “Device or Diagnosing Male Infertility,” filed Sep. 1, 2021, which is incorporated herein, in its entirety, by this reference.

### FIELD OF THE INVENTION

This disclosure relates generally to diagnostic devices for diagnosing male infertility. In particular this disclosure relates to a diagnostic device including a thermally conductive probe configured to contact a Scrotal-Testes (ST) Complex of a patient.

### BACKGROUND OF THE INVENTION

Presently, there is no diagnostic device available to identify male infertility even though about 35% of couples with infertility may have a contributory male factor and in about 8% of couples a male factor is solely responsible for the couple's infertility. Many of these men have no identifiable cause for their infertility. It is possible that any subtle alteration in spermatogenesis may affect spermatozoon quality in a manner that cannot be detected by standard sperm analysis or currently available diagnostic tests. Therefore, newer diagnostic methods need to be developed.

In most mammals spermatogenesis requires about 2.0 to 2.5° C. lower testicular temperature than core body temperature. The Scrotal Testis (ST) Complex naturally has dynamic temperature regulation capabilities to achieve this cooler temperature. The scrotum, which houses the testis, has no subcutaneous fat, thereby allowing for the cooling of the testes in conjunction with the changing surface area of the scrotal wall. In addition, the spermatic cord houses counter-current exchange between incoming arterial blood and outgoing venous blood, which pre-cools the arterial blood arriving at the testis. Any deviation in this system such as that which occurs with varicocele will alter the testicular temperature and may have deleterious effects on spermatogenesis.

Human testicular biopsies from patients with varicocele, cryptorchidism, and retractile testes have architectural sloughing with immature cells, spermatogenic arrest, germinal cell hypoplasia, peritubular fibrosis, apical cytoplasmic degeneration of Sertoli cells, and vacuolated Leydig cells.

Transient heat shock in mice causes a temporary reduction in testis weight and decreased sperm concentration and motility, with partial or complete infertility. Female mice mated with males who experienced transient heat shock showed a decrease in the rate of in-vitro fertilization and had poor in-vivo pregnancy rates. Spermatocytes are apparently particularly susceptible to elevated temperatures. In addition, the few motile spermatozoa remaining after heat shock were found to have damaged DNA.

Human volunteers who became either azoospermic or oligozoospermic following induced hyperthermia (by tightly suspending the ST Complex) recovered to pre-suspension levels within six months after the end of the study. Conversely, men with poor semen quality who were exposed to scrotal cooling for 12 weeks significantly increased their sperm concentration and output. Thus, there is evidence to suggest that hyperthermia of the ST Complex causes sub-fertility or infertility.

Absolute scrotal temperature is not known, since a method or technique for scrotal temperature measurement has not been standardized. Nonetheless, the ranges of human scrotal temperatures reported using many different methods range from 28 to 35° C.

The ST Complex temperature has been measured by many different methods, producing differing results. These methods may not provide a true representative temperature of the ST Complex, since various regions of the gonad are known to be at different temperatures. For example, a temperature difference of 1.9° C. between the anterior and posterior regions of the testis has been reported.

Also, only a slight increase in mean testis temperature (0.4 to 0.7° C.) between infertile and fertile men has been reported. Relying on such small temperature differences for the evaluation of individual patients is therefore impractical and problematic. Magnifying these relatively minute temperature differences to assess overall spermatogenetic temperature may, however, prove helpful.

### SUMMARY

Diagnostic devices for diagnosing male infertility and associated methods are described herein. In one aspect, a diagnostic device can include a thermally conductive probe configured to contact a Scrotal-Testes (ST) Complex of a patient; a thermally insulative housing detachably coupled to the thermally conductive probe; and a base defining a cavity, the base including: a thermal sensor positioned within the cavity and detachably coupled to a bottom surface of the thermally conductive probe; at least one processor in electronic communication with the thermal sensor and positioned within the cavity; and a receptacle formed by an exterior surface of the base and detachably coupled to the thermally insulative housing.

This aspect can include a variety of embodiments. In one embodiment, the receptacle can further include a lip configured to receive the thermally insulative housing. In some cases, the lip can define one or more notches configured to receive corresponding protrusions of the thermally insulative housing.

In another embodiment, the thermally insulative housing can include one or more protrusions formed on an axial surface of the thermally insulative housing. In some cases, the one or more protrusions can include two or more protrusions, where the two or more protrusions are equidistance with respect to each other.

In another embodiment, the thermally conductive probe can be composed of 316 stainless steel.

In another embodiment, the thermally insulative housing can be composed of Polytetrafluoroethylene (PTFE), Nylon, Teflon, and the like.

In another embodiment, the thermal processor can include a thermocouple.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a perspective view of a diagnostic device for diagnosing male infertility according to an embodiment of the present disclosure.

FIG. 2 depicts an isometric view of a probe for a diagnostic device according to an embodiment of the present disclosure.

FIG. 3 depicts a side cross-sectional view of a probe for a diagnostic device according to an embodiment of the present disclosure.

FIG. 4A, FIG. 4B, and FIG. 4C each depict separated and assembled components of a probe for a diagnostic device according to an embodiment of the present disclosure.

FIGS. 5 and 6 depict images of diagnostic devices for treating male infertility according to embodiments of the present disclosure.

FIG. 7A, FIG. 7B, and FIG. 7C each depict cross-sectional views of a diagnostic device according to embodiments of the present disclosure. FIG. 7A depicts a side cross-section view of a base of a diagnostic device. FIG. 7B depicts a top cross-sectional view of a housing of a diagnostic device. FIG. 7C depicts a side cross-sectional view of a housing and probe of a diagnostic device.

FIG. 8 depicts an image of a diagnostic device for treating male infertility, including various dimensions, according to an embodiment of the present disclosure.

FIG. 9 depicts a process flow for diagnosing male infertility according to an embodiment of the present disclosure.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

A diagnostic device for diagnosing male infertility are described herein. The device can measure the amount of heat generated by the ST Complex. The diagnostic device can offer a valuable service to infertility patients by utilizing state-of-the-art technology to economically and non-invasively diagnose male infertility. The diagnostic device can evaluate fertility potential by reliably measuring the radiated heat by the ST Complex under physiological stress conditions. Physiological stress has been known to amplify subtle defects that are not usually visible or detectable under normal situations (e.g., physical stress test for cardiovascular assessment). If the measurements are higher than the standard established for fertile males, it enables the diagnosis of heat-induced sub-fertility or infertility.

The diagnostic device can accurately and non-invasively identify men with a higher ST Complex temperature, thereby offering a unique opportunity to diagnose sub-fertility or infertility due to higher than average testicular temperature.

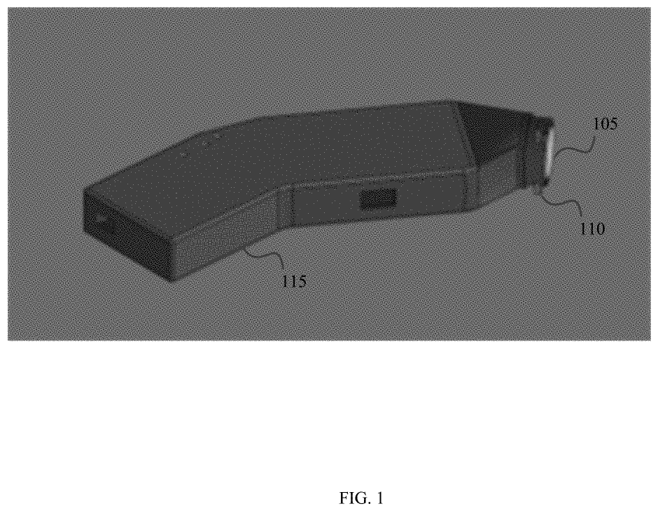

As depicted in FIG. 1, the diagnostic device can include a probe 105, a housing 110, and a base 115. The probe 105 can be configured for contacting a portion of a ST Complex of a patient, and measuring temperature from the ST Complex portion. The probe 105 can be made of a thermally conductive composition. Examples of such compositions can include silver, copper, gold, silicon carbide, aluminum, tungsten, steel, and the like.

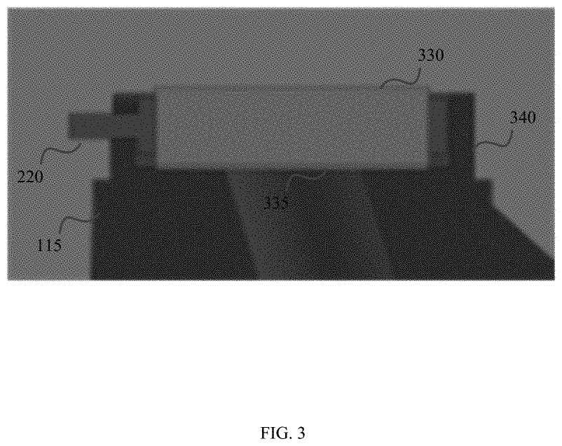

The probe 105 can also include dimensions sufficient for contacting a particular portion of the ST Complex surface. For example, the probe 105 can include an overall length and width sufficient for contacting the testicular surfaces of one or both testicles. Further, the thickness of the probe 105 can be configured for optimal thermal conductivity between the probe surface (e.g., surface 330 of FIG. 3) for contacting the patient's ST Complex, and the surface (e.g., surface 335 of FIG. 3) contacting a thermal sensor housed in the base. The thickness of the probe 105 can be sufficient so as to mitigate thermal dissipation through the probe, mitigate ambient temperature effects on the probe 105, and the like.

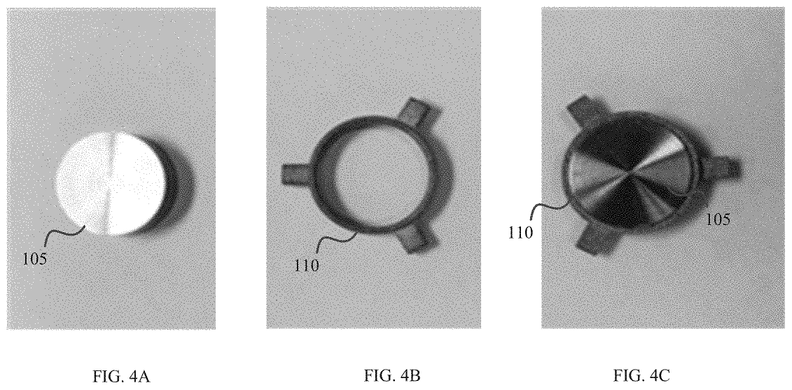

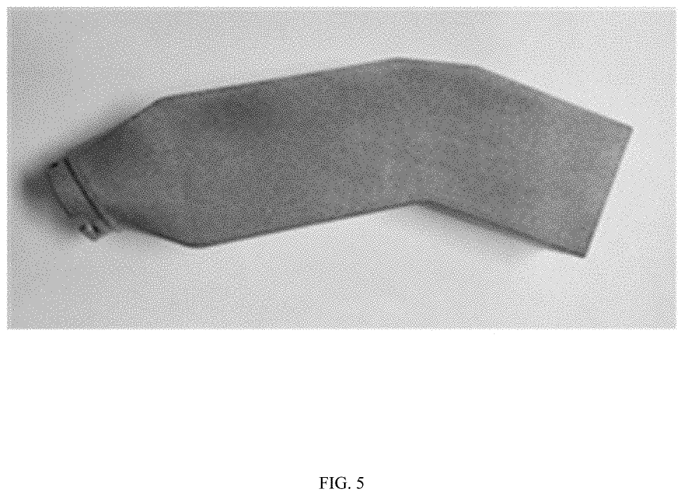

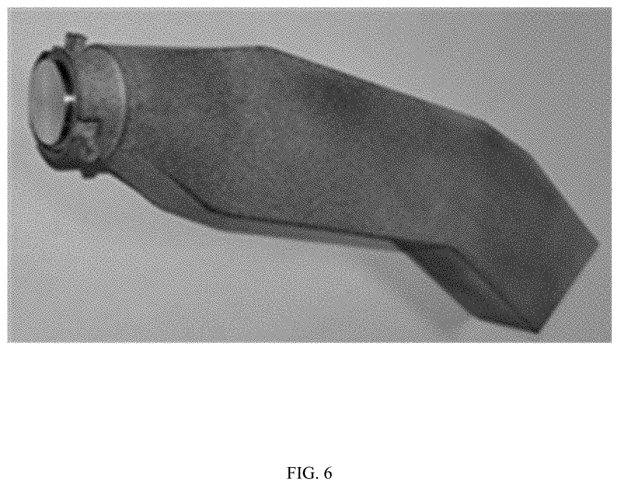

The diagnostic device can also include a housing 110. The housing 110 can be configured to detachably couple the probe 105 to the base 115 of the diagnostic device. For example, the housing 110 can define an aperture configured to receive the probe 105 within the housing 110. The aperture can be configured such that, when the probe 105 is coupled to the housing 110, the probe surface for contacting the ST Complex (e.g., surface 330) is exposed from the housing, and the probe surface for contacting or coupling to the thermal sensor of the base (e.g., surface 335) is exposed from the housing 110. This embodiment is illustrated in FIG. 3. In the examples provided in FIGS. 1-3, 4A, 4B, 4C, and 5-6, the housing 110 is in the shape of a ring, where the interior annular surface of the housing is configured for receiving the probe disc. Further, the width dimension of the housing 110 can be slightly smaller than the thickness dimension of the probe 105, such that when the probe 105 is received in the housing 110, the probe surface 330 protrudes slightly from the housing 110.

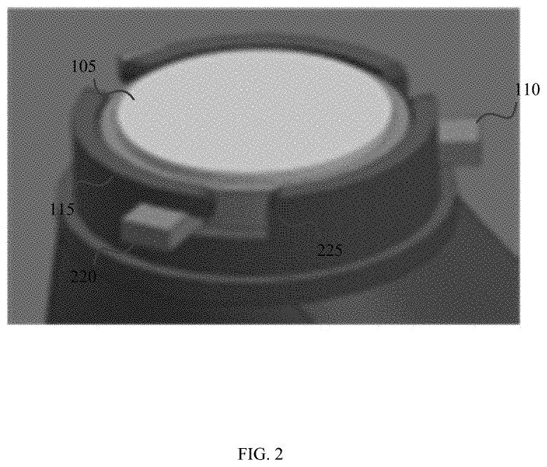

The housing 110 can also define one or more protrusions or notches for securing the housing to the base. For example, as shown in FIG. 2, the housing 110 can define a set of protrusions 220 protruding from the radial surface (e.g., defined by the housing thickness) of the housing 110. The protrusions 220 can be configured to couple to corresponding notches 225 defined by the base 115 to secure the housing 110 to the base 115. Alternatively, the housing 110 can define a set of notches configured to couple to corresponding protrusions defined by the base 115. The figures of the present disclosure depicts the housing having three radial protrusions equidistant from each other, however one skilled in the art will understand that the number, size, and positioning of the protrusions can vary.

The housing 110 can be composed of a thermally insulating material. For example, the housing 110 can be composed of Nylon PA12, Polytetrafluoroethylene (PTFE), Polyurethane, Poly Vinyl Chloride (PVC), Silicone Rubber, ceramics, PolyEtherEther-Ketone (PEEK), and the like.

The diagnostic device can also include a base 115. The base 115 can include an exterior shell defining a cavity. The cavity can house integrated hardware and software for receiving temperature measurements from, processing the temperature measurements, and communicating the raw and processed data to external sources. For example, the base 115 may house one or more sensor driver configured to receive measurement data from the probe 105. The base 115 may also house one or more processors configured to process the measurement data. In other cases, the device may offload the processing to external computing devices or cloud computing entities.

The base 115 can also define a receptacle 340 configured to receive the housing 110. For example, the base 115 can form a lip in the shape of the housing 110. The lip can extend from the body of the base 115, where the extending dimension can be the same size as the width of the housing 110 (e.g., such that the housing top surface is flush with the distal edge of the lip when positioned in the receptacle). Further, the receptacle 340 can define one or more notches in the lip. The corresponding notches can correspond to the protrusions formed by the housing 110, and can secure the housing 110 to the base 115. In some cases, the notches can be “L” notches, such that the housing 110 is first compressed onto the receptacle, and then twisted to secure the housing 110 to the base 115.

The receptacle 340 can also define an aperture for accessing the cavity of the body 115. The aperture may be defined by a bottom surface of the receptacle 340. Alternatively, the aperture may be defined by the exterior shell or the receptacle lip (e.g., no bottom surface of the receptacle is provided). The aperture can provide access to the internal components of the base 115 to the probe 105 and housing 110. For example, the probe 105 can be coupled to a temperature sensor (e.g., via physical contact, a thermocouple sensor, or non-physical contact with an infrared sensor) through the aperture when the probe 105 and housing 110 are secured to the base 115. In some cases, the temperature sensor may include a contact surface for coupling to the probe 105. The contact surface may be positioned flush with the aperture, such that the contact surface is in physical contact or non-physical contact with the bottom surface 335 of the probe when the probe 105 and housing 110 are coupled to the base 115.

In a particular embodiment, the diagnostic device can include a probe composed of 20 mm disk of 316 stainless steel (specific heat 500) weighing 15 grams in total, housed in 1 mm thick Nylon with one surface exposed. An infrared sensor is placed opposite to the exposed surface, but inside the Nylon housing. The infrared sensor is connected to a data acquisition system that communicates with a laptop computer containing the appropriate software program.

### Process

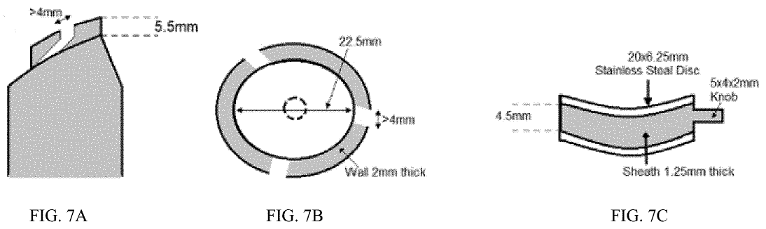

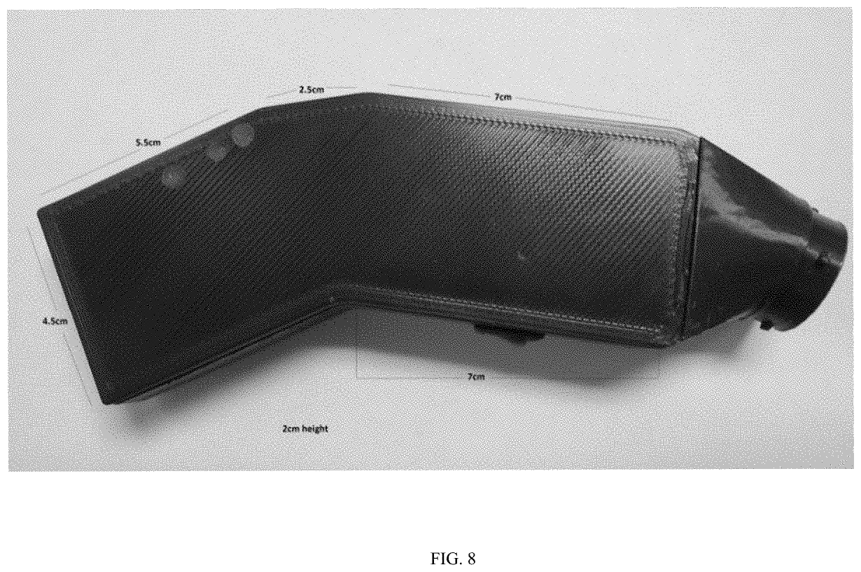

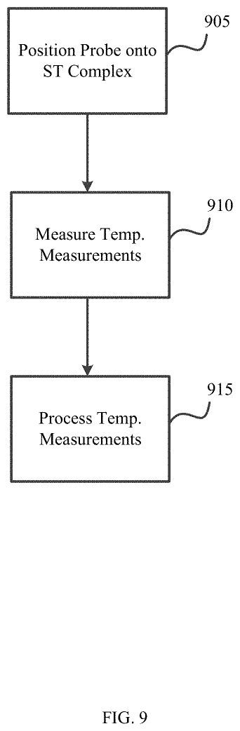

FIG. 9 depicts a process flow for diagnosing male infertility according to an embodiment of the present disclosure. The process flow can be implemented at least in part by a diagnostic device, such as the diagnostic devices described above with reference to FIGS. 1-3, 4A, 4B, 4C, 5-6, 7A, 7B, 7C, and 8.

The probe of the diagnostic device can be cooled to a predefined threshold (e.g., 0° C.). The cooled probe can be locked into position onto the base receptacle via the housing. The exposed surface of the probe can then be positioned on an ST Complex of a patient (Step 905). The diagnostic device can measure temperature measurements while the exposed surface of the probe is in contact with the ST Complex (Step 910). For example, the diagnostic device can measure temperature in predefined time increments (e.g., increments of between 1 to 5 seconds for a total of 10 minutes). The diagnostic device can also process the temperature measurements to diagnose male infertility of the patient (Step 915). For example, the diagnostic device can calculate heat generated by the ST Complex. The process can be repeated on the other ST Complex of the patient.

### Application

The diagnostic device described herein can be implemented for diagnosing a variety of ailments. While the device described above is configured for diagnosing male infertility, the device can be configured for measuring the temperature of other body parts of a patient for other particular diagnoses. For example, the diagnostic device can be used for: diagnosis of the extent of muscular or ligament injury; detection of the extend of inflammation in rheumatology; detection of the extent of vascular irregularity; screening for the detection of breast cancer; and the like.

### Experimental Discussion

A preliminary study with sixteen men attending an infertility clinic was conducted to determine the ST Complex temperature. Eight of these sixteen men had low sperm concentration (<20×10⁶/ml) and the remaining eight had normal sperm concentration (>20×10⁶/ml). The participants volunteered to have their ST Complexes immersed in 150 to 180 ml of water at 5° C. The increase in water temperature was then monitored for 10 to 12 minutes.

Heat generated per unit volume of testicle per unit time was calculated as a fraction of the volume of water to that of the volume of testicles (determined with a Prader orchidometer), multiplied by the specific heat of water, multiplied by the difference between ending and starting temperatures of the water. For convenience, this value will be referred to as the ST Complex Heat Index. The semen quality and the ST Complex Heat Index for both groups of men are listed in Table 1.

TABLE 1
Semen Quality and ST Complex Heat Index mean ± SD values
for men having normal sperm concentration (>20 ×
10⁶/ml) and men having low sperm concentration (<20 × 10⁶/ml).

| | Men with >20 × | Men with <20 × |
|---|---|---|
| Variables | 10⁶/ml Sperm | 10⁶/ml Sperm |
| Semen Volume (ml) | 4.3 ± 1.9 | 5.1 ± 1.8 |
| Sperm Concentration (×10⁶/ml)* | 130.0 ± 140.1 | 6.1 ± 2.6 |
| Sperm Motility (%) | 50.1 ± 12.4 | 36.5 ± 17.9 |
| Sperm Morphology (%)* | 21.6 ± 9.2 | 11.6 ± 8.6 |
| ST Complex Heat Index** | 70.8 ± 9.0 | 111.4 ± 25.6 |

*Significantly Different (p < 0.05) from each other;
**Significantly Different (p < 0.001) from each other.

As expected, the sperm concentrations between the two groups of men are significantly different, since they were selected for the study based on the differences in sperm numbers. Though the sperm morphology was also significantly different between the two groups, it alone or in conjunction with other sperm parameters cannot convincingly identify male infertility.

Interestingly, not only was the ST Complex Heat Index significantly different (p<0.001), but the individual values clustered at the extreme opposite ends between the two groups of men (Table 2) also varied considerably. In at least five men in the low sperm concentration group, hyperthermia of the testis was a contributory cause, if not the primary cause.

TABLE 2
Distribution of ST Complex Heat Index for
men with normal or low sperm concentration.

| | | | |
|---|---|---|---|
| ST Complex | 150 | | |
| Heat Index | 140 | | ● |
| | | | ● |
| | 130 | | ● |
| | 120 | | |
| | | | ●● |
| | 110 | | |
| | 100 | | |
| | | | ● |
| | 90 | | ● |
| | | ● | |
| | 80 | ● | |
| | | ●● | |
| | 70 | ● | ● |
| | | ●● | |
| | 60 | | |
| | | ● | |
| | 50 | | |
| | | >20 × 10⁶/ml | <20 × 10⁶/ml |

Sperm Concentration

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A diagnostic device for treating male infertility comprising:
a thermally conductive probe configured to contact a Scrotal-Testes (ST) Complex of a patient;
a thermally insulative housing detachably coupled to the thermally conductive probe; and
a base defining a cavity, the base comprising:
a thermal sensor positioned within the cavity and detachably coupled to a bottom surface of the thermally conductive probe;
at least one processor in electronic communication with the thermal sensor and positioned within the cavity; and
a receptacle formed by an exterior surface of the base and detachably coupled to the thermally insulative housing.

2. The diagnostic device of claim 1, wherein the receptacle further comprises a lip configured to receive the thermally insulative housing.

3. The diagnostic device of claim 2, wherein the lip defines one or more notches configured to receive corresponding protrusions of the thermally insulative housing.

4. The diagnostic device of claim 1, wherein the thermally insulative housing comprises one or more protrusions formed on an axial surface of the thermally insulative housing.

5. The diagnostic device of claim 4, wherein the one or more protrusions comprise two or more protrusions, wherein the two or more protrusions are equidistance with respect to each other.

6. The diagnostic device of claim 1, wherein the thermally conductive probe is composed of 316 stainless steel.

7. The diagnostic device of claim 1, wherein the thermally insulative housing is composed of Polytetrafluoroethylene (PTFE), Nylon, Teflon, or a combination thereof.

8. The diagnostic device of claim 1, wherein the thermal processor comprises an infrared sensor.
